(19) United States
(12) Patent Application Publication
(10) Pub. No.: US 2005/0119477 A1
(43) Pub. Date: Jun. 2, 2005

(54) Method for determining specific groups constituting heparins or low molecular weight heparins

(75) Inventors: Pierre Mourier, Charenton Le Pont (FR); Christian Viskov, Ris Orangis (FR)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Aventis Pharma S.A., Antony Cedex (FR)

(21) Appl. No.: 10/665,872

(22) Filed: Sep. 18, 2003

Related U.S. Application Data
(60) Provisional application No. 60/422,482, filed on Oct. 31, 2002.

(30) Foreign Application Priority Data
Sep. 23, 2002 (FR) 02 11724

(51) Int. Cl. C07H 1/00

(52) U.S. Cl. 536/123.1

(57) ABSTRACT

The subject of the invention is a method for analysing heparins or low-molecular-weight heparins, characterized in that the sample to be assayed is depolymerized by the action of heparinases and then, where appropriate, the depolymerizate obtained is reduced and then an analysis is carried out by high performance liquid chromatography.

This application claims the benefit of U.S. Provisional Application No. 60/422,482 filed Oct. 31, 2002, and right of priority from French Patent Application No. 02 11724, filed Sep. 23, 2002.

The subject of the present invention is a method for analysing specific groups constituting heparins or low-molecular-weight heparins.

During the process for preparing enoxaparin (Lovenox®) (U.S. Pat. No. 5,389,618) from pure heparin, the aqueous-phase alkaline depolymerization process produces a partial but characteristic conversion of the glucosamines of the reducing ends of the oligosaccharide chains.

The first step of this conversion consists of a glucosamine⇄mannosamine epimerization (T. Toida et al., J. Carbohydrate Chemistry, 15(3), 351-360 (1996)); the second step is a 6-O-desulfation of the glucosamine, leading to the formation of derivatives called “1,6 anhydro” (international patent application WO 01/29055).

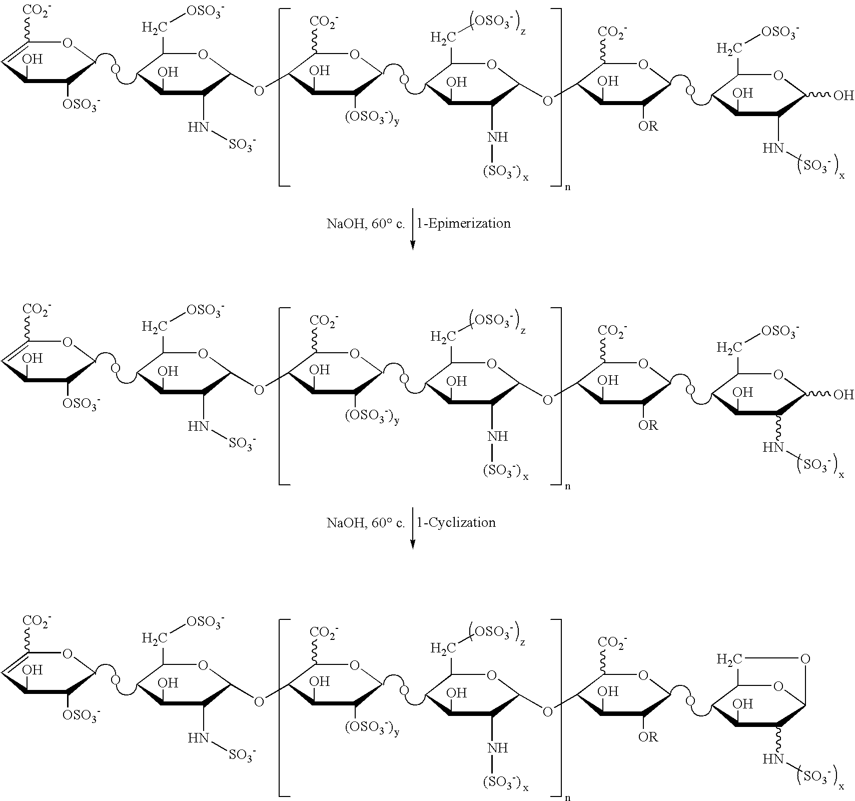

This type of derivative is only obtained for oligosaccharide chains whose terminal glucosamine is 6-O-sulfated.

The percentage of oligosaccharide chains whose end is modified with a 1,6-anhydro bond is a structural characteristic of the oligosaccharide mixture of Lovenox and it should be possible to measure it.

The present invention therefore consists of a method for analysing heparins, low-molecular-weight heparins and more particularly Lovenox.

The method of analysis according to the invention is the following:

The sample to be assayed is depolymerized by the action of heparinases and then, where appropriate, the depolymerizate obtained is reduced and then analysis is carried out by high-performance liquid chromatography.

The method as defined above is therefore characterized in that there is a search for the presence of oligosaccharide chains whose end is modified with a 1,6-anhydro bond (“1,6-anhydro groups”).

In particular, the sample to be assayed is first of all exhaustively depolymerized with a mixture of heparinases and in particular heparinase 1 (EC 4.2.2.7.), heparinase 2 (heparin lyase II) and heparinase 3 (EC 4.2.2.8.). (These enzymes are marketed by the group Grampian Enzymes).

The subject of the invention is therefore a method for analyzing heparins or low-molecular-weight heparins, characterized in that the following steps are carried out:
1) depolymerization of the sample by the action of heparinases
2) where appropriate, reduction of the depolymerizate
3) assay by high-performance liquid chromatography.

The subject of the invention is more particularly the method as defined above, characterized in that the heparinases are in the form of a mixture of heparinase 1 (EC 4.2.2.7.), heparinase 2 (heparin lyase II) and heparinase 3 (EC 4.2.2.8.).

The depolymerizate thus prepared is then treated preferably with an NaBH₄ solution in sodium acetate. The latter operation makes it possible to specifically reduce the reducing ends which are not in the 1,6-anhydro form (products described in patent application WO 01/72762). Finally, in order to be able to quantify the disaccharides 1 and 2 described below, the sample of low-molecular-weight heparin, depolymerized with heparinases, should be reduced by the action of a reducing agent such as NaBH₄.

The subject of the invention is therefore more particularly the method as defined above, characterized in that the depolymerized heparin is then reduced.

The subject of the invention is most particularly the method as defined above, characterized in that the reducing agent is NaBH₄. Another alkali metal salt of borohydride such as lithium or potassium may be optionally used.

The assay of the 1,6-anhydro ends is then carried out by HPLC (High Performance Liquid Chromatography) and in particular by anion-exchange chromatography.

The method of assay according to the invention makes it possible to clearly differentiate Lovenox from the other low-molecular-weight heparins which do not contain these “1,6-anhydro” derivatives. Conversely, the method of assay according to the invention makes it possible to ascertain that low-molecular-weight heparins do not satisfy the physicochemical characteristics of Lovenox and therefore are different in nature.

The method of assay according to the invention may be applied to the industrial process during in-process control of samples in order to provide standardization of the process for manufacturing Lovenox and to obtain uniform batches.

After enzymatic depolymerization and reduction of the reducing ends, the 1,6-anhydro derivatives of Lovenox exist in 4 essential forms. The subject of the invention is therefore also the method as described above, characterized in that the 1,6-anhydro residues obtained during the depolymerization reaction are the following:

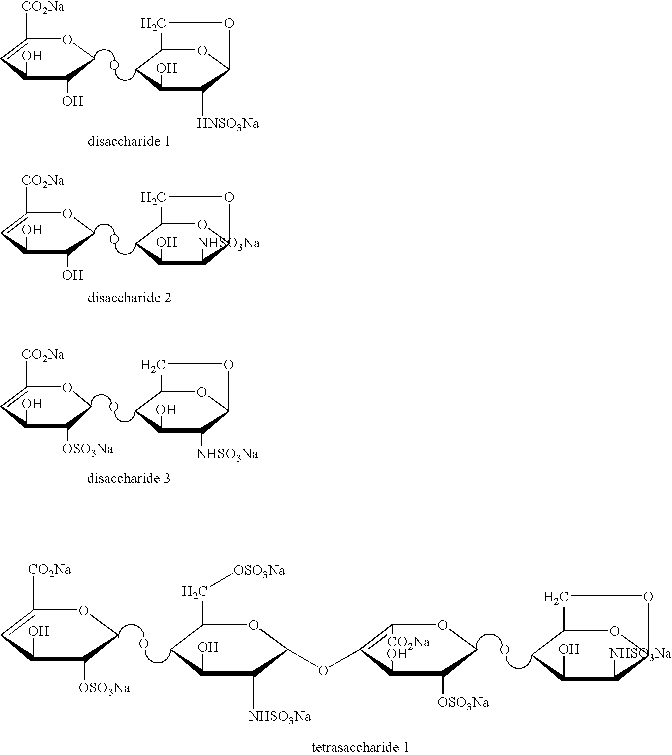

All the oligosaccharides or polysaccharides which contain the 1,6-anhydro end on the terminal disaccharide unit and which do not possess a 2-O-sulfate on the uronic acid of said terminal disaccharide are completely depolymerized by the heparinases and in the form of the disaccharides 1 and 2. On the other hand, when said terminal saccharide contains a 2-O-sulfate on the uronic acid and when it is in the mannosamine form, the 1,6-anhydro derivative is in the form of the tetrasaccharide 1 (form resistant to heparinases).

The trisaccharide 1 (see below) is also present in the mixture. It is derived from another degradation process 20 which leads to the structure below (peeling phenomenon observed during the chemical depolymerization of Lovenox).

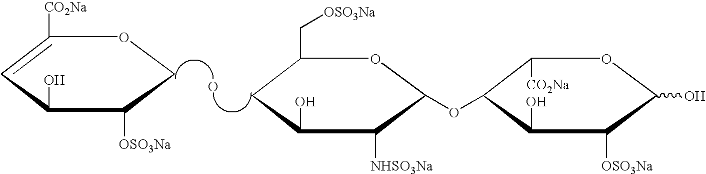

The other constituents of the mixture are not characteristic solely of Lovenox. There are of course the 8 elementary disaccharides of the heparin chain. These 8 elementary disaccharides are marketed inter alia by the company Sigma.

Other disaccharides were identified in the mixture by the method according to the invention: the disaccharides ΔIIs_{gal} and ΔIVS_{gal} which have as origin alkaline 2-O-desulfation of -IdoA(2S)-GlcNS(6S)- and of -IdoA(2S)-GlcNS-, leading to the formation of 2 galacturonic acids. They are not usually present in the original structure of heparin (U. M. Desai et al., Arch. Biochem. Biophys., 306 (2) 461-468 (1993).

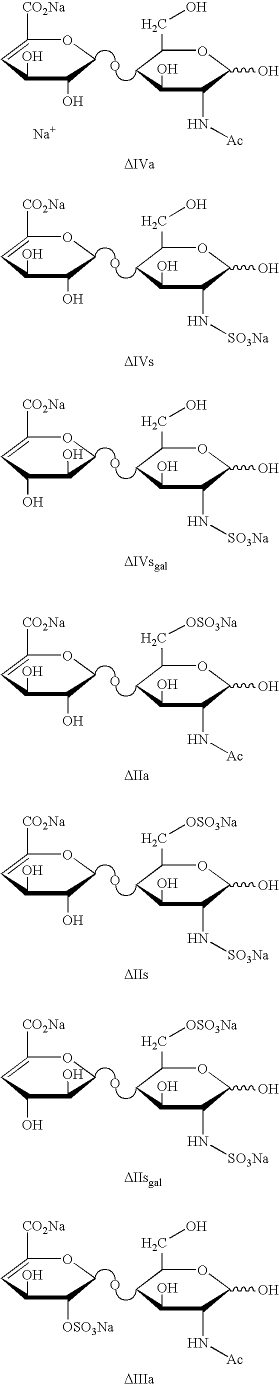

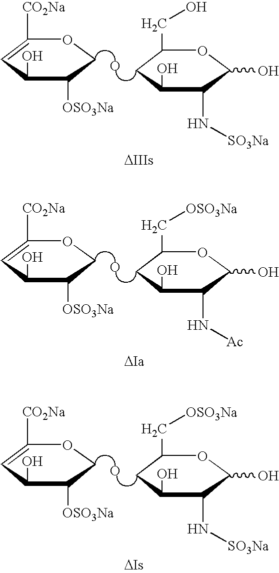

The oligosaccharides containing 3-O-sulfated glucosamines withstand cleavage by heparinases and remain present in the form of tetrasaccharides.

In the case of most low-molecular-weight heparins, the heparin is extracted from pig mucus, and these principal tetrasaccharides are represented below. They are resistant to enzymatic depolymerization and reflect the sequences with affinity for antithrombin III. They are symbolized as follows: ΔIIa-IIs_{glu} and ΔIIa-IVs_{glu}. (S. YAMADA, K. YOSHIDA, M. SUGIURA, K-H KHOO, H. R. MORRIS, A. DELL, J. Biol. Chem.; 270(7), 4780-4787 (1993)

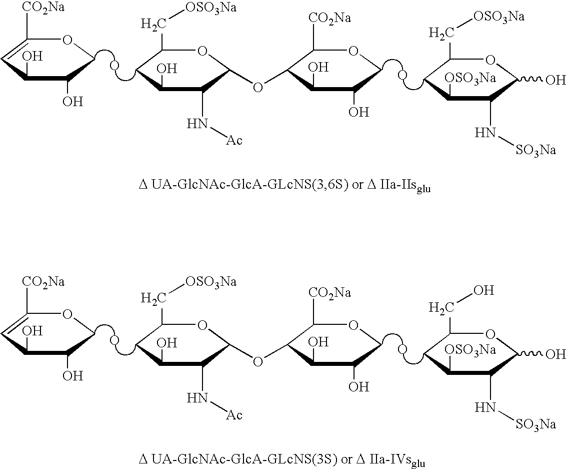

The final constituent of the mixture cleaved with heparinases is the glycoserine end ΔGlcA-Gal-Gal-Xyl-Ser (K. SUGAHARA, H. TSUDA, K. YOSHIDA, S. YAMADA, J. Biol. Chem.; 270(39), 22914-22923 (1995); K. SUGAHARA, S. YAMADA, K. YOSHIDA, P. de WAARD, J. F. G. VLIEGENTHART; J. Biol. Chem.; 267(3), 1528-1533 (1992). The latter is generally almost absent from Lovenox (see NMR in Example 5).

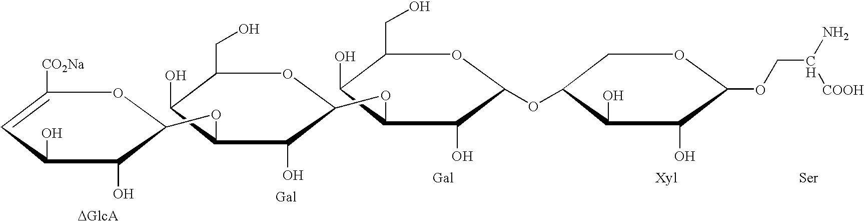

Another aspect of the invention consists in the chromatography process used for determining the 1,6-anhydro groups. First of all, it involves separating the various polysaccharides obtained after depolymerization and treatment with a reducing agent such as NaBH₄.

Anion-exchange chromatography (SAX) is the separating method which is most suitable for such a complex mixture.

Columns filled with a stationary phase of the Spherisorb SAX type having a particle size of 5 μm and a length of 25 cm can be used. All the conventional column diameters between 1 mm and 4.6 mm can be used.

The equipment used may be a chromatograph allowing the formation of an elution gradient with a UV detector, more preferably equipped with an array of diodes in order to be able to produce UV spectra of the constituents and to record complex signals, resulting from the difference between the absorbance at 2 different wavelengths and allowing the specific detection of acetylated oligosaccharides. To allow this type of detection, mobile phases which are transparent in the UV region up to 200 nm are preferable. This excludes conventional mobile phases based on NaCl which have moreover the disadvantage of requiring a passivated chromatograph in order to withstand the corrosive power of the chlorides. The mobile phase used here will be preferably based on sodium perchlorate, but methanesulfonate or phosphate salts may also be used.

The pH recommended for the separation is from 2 to 6.5. Preferably, a pH in the region of 3 will be used. It is controlled here by adding a salt such as phosphate possessing a buffering power at pH=3 which is better than that of perchlorates.

By way of example, standard chromatographic separation conditions are given below:

| | |
|---|---|
| Solvent A: | NaH₂PO₄, 2.5 mM, brought to pH 2.9 by |
| | addition of H₃PO₄ |
| Solvent B: | NaClO₄ 1N- NaH₂PO₄, 2.5 mM, brought to |
| | pH 3.0 by addition of H₃PO₄ |

The elution gradient may be the following:
T=0 min: % B=3; T=40 min: % B=60; T=60 min: % B=80

The subject of the present invention is therefore also a method of analysis as defined above by separation by anion-exchange chromatography, characterized in that the mobile phase which is transparent in the UV region up to 200 nM is used.

The subject of the invention is more particularly a mobile phase as defined above based on sodium perchlorate, methanesulfonate salts or phosphate salts.

Another most important aspect consists in the method of detection.

A method is developed in order to increase the specificity of the UV detection. As nonacetylated polysaccharides all have, at a given pH, a fairly similar UV spectrum, it is possible to selectively detect the acetylated sugars by taking as signal the difference between the absorbance at 2 wavelengths chosen such that the absorptivity of the nonacetylated saccharides cancels out.

In the case below, 202 nm and 230 nm will be chosen as detection and reference wavelengths and the 202-230 nm signal will be noted. The choice of course depends on the pH of the mobile phase (adjustments of a few nm may be necessary so as to be at the optimum of said conditions). The most suitable detector for this technique is the DAD 1100 detector from the company Agilent Technologies. In this case, a double detection will be carried out at 234 nm, on the one hand, and at 202-230 nm, on the other hand. The principle of selective detection of acetylated oligosaccharides is illustrated in FIG. 1 in which the UV spectrum of a sulfated disaccharide Delta 1s is compared with that of an acetylated disaccharide Delta 1a.

The subject of the present invention is therefore also a method of analysis as defined above by separation by anion-exchange chromatography, characterized in that the method of detection makes it possible to selectively detect acetylated sugars.

The subject of the invention is also most particularly a method of analysis as defined above by separation by exchange chromatography, characterized in that the selective detection of acetylated sugars is carried out taking as signal the difference between the absorbance at 2 wavelengths chosen such that the absorptivity of the nonacetylated saccharides cancels out.

The quantification of the 4 1,6-anhydro residues described above requires a sufficient selectivity of the chromatographic system in relation to all the other constituents of the mixture. However, the 2 disaccharides 1 and 2, which are coeluted in general, are poorly resolved with respect to ΔIIa, especially as the latter is present in the form of its 2 α and β anomers.

The identity of the 2 disaccharides 1 and 2 may be easily verified because they form in a few hours at room temperature in an aqueous solution of ΔIIs brought to pH 13 by addition of NaOH. However, if double detection is used, the acetylated oligosaccharides ΔIVa, ΔIIa, VIIIa, ΔIa, VIIa-IVs_{glu} and ΔIIa-IIs_{glu} are easily identifiable.

The causes of splitting of the peaks are the anomeric forms, on the one hand, and to a lesser degree the glucosamine⇄mannosamine epimerization which is partially present for ΔIIs, ΔIIIs and ΔIs when they are in the terminal position in the oligosaccharide chain.

In order to be able to quantify the disaccharides 1 and 2, the sample of low-molecular-weight heparin, depolymerized by heparinases is reduced by the action of NaBH₄.

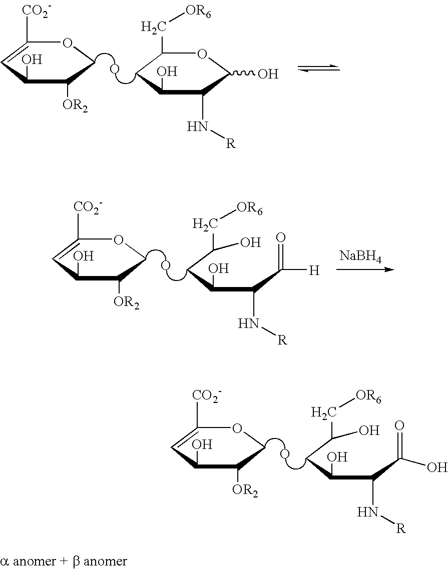

This reduction has the advantage of eliminating the α⇄β anomerisms by opening of the terminal oligosaccharide ring. The chromatogram obtained is simpler since the anomerisms are eliminated and especially the reduction of ΔIIa reduces its retention on the column and allows easy assay of the disaccharides 1 and 2.

The examples of chromatograms described in FIGS. 2 and 3 clearly illustrate these phenomena and the advantages of this method.

Finally, the subject of the invention is also the novel saccharide derivatives obtained using the depolymerization and reduction process, chosen from disaccharide 1, disaccharide 2, disaccharide 3 and trisaccharide 1.

### BRIEF DESCRIPTION OF THE DRAWINGS

FIG. 1 illustrates the selective detection of acetylated oligosaccharides in which the UV spectrum of a sulfated disaccharide Delta is 1s compared with that of an acetylated disaccharide Delta 1a.

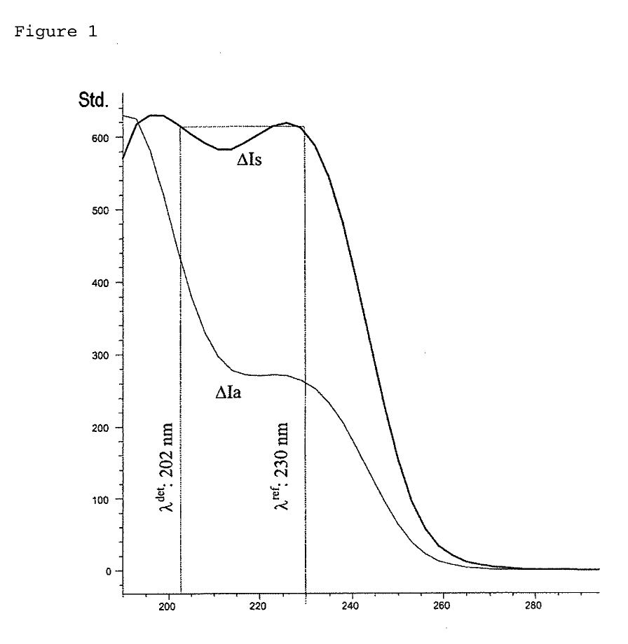

FIG. 2 shows the chromatographic separation of enoxaparin depolymerized with heparinases before and after reduction with NaBH₄ (signal in fine black: UV at 234 nm; signal in thick black: UV at 202-234=m)

FIG. 3 shows the chromatographic separation of heparin depolymerized with heparinases before and after reduction with NaBH₄ (signal in fine black: UV at 234 nm; signal in thick black: UV at 202-234 nm)

The examples below illustrate the invention without however having a limiting character.

### EXAMPLE 1

The enzymatic depolymerization is carried out for 48 hours at room temperature by mixing 50 μl of a solution containing 20 mg/ml of low-molecular weight heparin to be assayed, 200 μl of a 100 mM acetic acid/NaOH solution at pH 7.0 containing 2 mM calcium acetate and 1 mg/ml of BSA with 50 μl of the stock solution of the 3 heparinases.

The reduction is carried out on 60 μl of the product depolymerized with the heparinases by adding 10 μl of an NaBH₄ solution at 30 g/l in 100 mM sodium acetate prepared immediately before use. It will be noted that the heparinases are stored at −30° C. The heparinases are in a buffer solution and their titer is 0.5 IU/ml (composition of the buffer solution: aqueous solution pH 7 of KH₂PO₄ at a concentration of 0.01 mol/l and supplemented with bovine serum albumin (BSA) at 2 mg/ml).

### EXAMPLE 2

NMR of Disaccharide 3 obtained according to the process described above.

Proton spectrum in D₂O, 400 MHz, T=298K, δ in ppm: 3.34 (1H, dd, J=7 and 2 Hz, H2), 3.72 (1H, t, J=8 Hz, H6), 3.90 (1H, m, H3), 4.03 (1H, s, H4), 4.20 (1H, d, J=8 Hz, H6), 4.23 (1H, t, J=5 Hz, H3′), 4.58 (1H, m, H2′), 4.78 (1H, m, H5), 5.50 (1H, s, H1), 5.60 (1H, dd, J=6 and 1 Hz, H1′), 6.03 (1H, d, J=5 Hz, H4′)].

### EXAMPLE 3

NMR of the Tetrasaccharide 1 obtained according to the process described above.

Proton spectrum in D₂O, 400 MHz, T=298K, δ in ppm: 3.15 (1H, s, H2), 3.25 (1H, m, H2″), 3.60 (1H, m, H3″), between 3.70 and 4.70 (14H, unresolved complex, H3/H4/H6, H2′/H3′/H4′/H5′, H4″/H5″/H6′, H2′″/H3′″), 4.75 (1H, m, H5), between 5.20 and 5.40 (2H, m, H1′ and H1″), 5.45 (1H, m, H1′″), 5.56 (1H, m, H1), 5.94 (1H, d, J=5 Hz, H4)

### EXAMPLE 4

NMR of the Trisaccharide 1 obtained according to the process described above.

Spectrum in D₂O, 600 MHz, (δ in ppm): 3.28 (1H, m), 3.61 (1H, t, 7 Hz), 3.79 (1H, t, 7 Hz), 3.95. (1H, d, 6 Hz), 4.00 (1H, s), 4.20 (1H, m), 4.28 (2H, m), 4.32 (1H, d, 4 Hz), 4.41 (1H, s), 4.58 (1H, s), 4.61 (1H, s), 4.90 (1H, broad s), 5.24 (1H, s), 5.45 (1H, s), 5.95 (1H, s).

### EXAMPLE 5

NMR of ΔGlcA-Gal-Gal-Xyl-Ser

Spectrum in D₂O, 500 MHz (δ in ppm): 3.30 (1H, t, 7 Hz), 3.34 (1H, t, 8 Hz), 3.55 (1H, t, 7 Hz), 3.60 (1H, t, 7 Hz), between 3.63 and 3.85 (10H, m), 3.91 (2H, m), 3.96 (1H, dd, 7 and 2 Hz), between 4.02 and 4.10 (3H, m), 4.12 (1H, d, 2 Hz), 4.18 (1H, m), 4.40 (1H, d, 6 Hz), 4.46 (1H, d, 6 Hz), 4.61 (1H, d, 6 Hz), 5.29 (1H, d, 3 Hz), 5.85 (1H, d, 3 Hz).

### EXAMPLE 6

### Principle of the Quantification

In the method according to the invention, the widely accepted hypothesis that all the unsaturated oligosaccharides contained in the mixture have the same molar absorptivity, equal to 5500 mol⁻¹.l.cm⁻¹ is made.

It is therefore possible to determine the percentage by weight of all the constituents of the depolymerized mixture in the starting low-molecular-weight heparin. For the 4 1,6-anhydro derivatives which correspond to the peaks 7, 8, 13 and 19, the following percentages by weight are obtained:

$$\% w / {w}_{7 + 8} = 100 \cdot \frac{443 \cdot ( {Area}_{7} + {Area}_{8} )}{\sum {Mw}_{x} \cdot {Area}_{x}} ;$$

$$\% w / {w}_{13} = 100 \cdot \frac{545 \cdot {Area}_{13}}{\sum {Mw}_{x} \cdot {Area}_{x}}$$

$$\% w / {w}_{19} = 100 \cdot \frac{1210 \cdot {Area}_{13}}{\sum {Mw}_{x} \cdot {Area}_{x}}$$

Area₇, Area₈, Area₁₃ and Area₁₉ correspond to the areas of each of the peaks 7, 8, 13 and 19. The molar masses of each of these 4 compounds are 443, 443, 545 and 1210 respectively. ΣMwₓ·Areaₓ corresponds to the ratio of the area of each peak of the chromatogram by the molar mass of the corresponding product.

If M_{w} is the mean mass of the low-molecular-weight heparin studied, the percentage of oligosaccharide chains ending with a 1,6-anhydro ring is obtained in the following manner:

$${\%}_{1.6 anhydro} = {M}_{W} \cdot ( \frac{\% w / {w}_{7 + 8}}{443} + \frac{\% w / {w}_{13}}{545} + \frac{\% w / {w}_{19}}{1210} )$$

The molecular masses of the constituents are the following:

| | Oligosaccharide | |
|---|---|---|
| Oligosaccharide | after reduction | Molecular mass |
| 1 | 1 | 741 |
| 2 | 20 | 401 |
| 3 | 3 | 734 |
| 4 | 21 | 461 |
| 5 | 22 | 461 |
| 6 | 23 | 503 |
| 7 | 7 | 443 |
| 8 | 8 | 443 |
| 9 | 24 | 503 |
| 10 | 25 | 563 |
| 11 | 26 | 563 |
| 12 | 27 | 563 |
| 13 | 13 | 545 |
| 14 | 28 | 605 |
| 15 | 29 | 1066 |
| 16 | 30 | 665 |
| 17 | 31 | 965 |
| 18 | 32 | 1168 |
| 19 | 19 | 1210 |

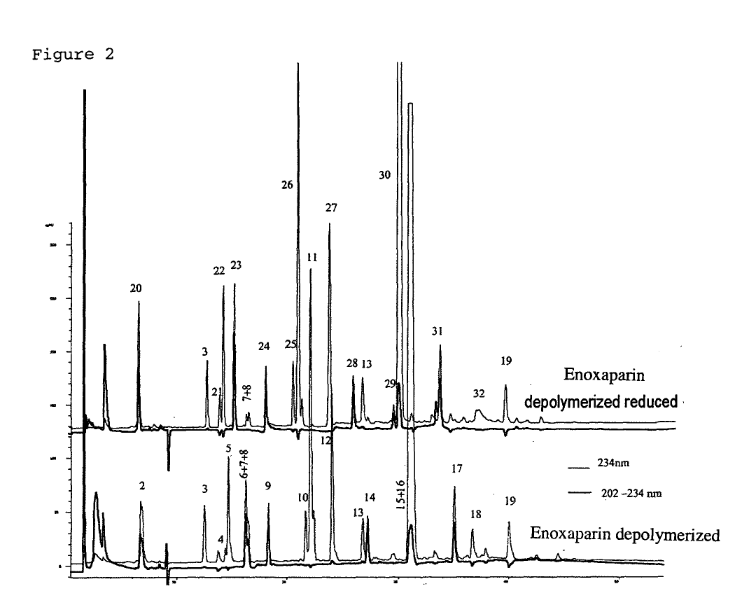

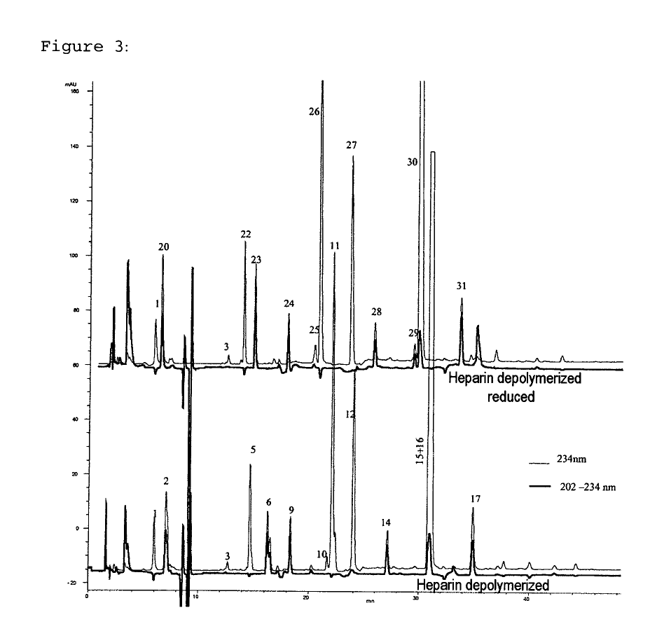

Nomenclauture of the Saccharides and Correspondence with the Peaks According to FIGS. 2 and 3
IdoA: α-Idopyranosyluronic acid;
GlcA: β-Glucopyranosyluronic acid;
ΔGlcA: 4,5-unsaturated acid: 4-deoxy-α-L-threo-hex-enepyranosyluronic acid;
Gal: D-Galactose;
Xyl: xylose;
GlcNAc: 2-deoxy-2-acetamido-α-D-glucopyranose;
GlcNS: 2-deoxy-2-sulfamido-α-D-glucopyranose;
2S: 2-O-sulfate,
3S: 3-O-sulfate,
6S: 6-O-sulfate
1: ΔGlcAβ₁₋₃ Gal β₁₋₃ Galβ₁₋₄ Xyl β₁-O-Ser
2: 4-deoxy-α-L-threo-hex-enepyranosyluronic acid-(1→4)-2-deoxy-2-acetamido-α-D-glucopyranosyl sodium salt
3: ΔGlcAβ₁₋₃ Gal β₁₋₃ Galβ₁₋₄ Xyl β₁-O—CH₂—COOH
4: 4-deoxy-α-L-threo-hex-4-enegalactopyranosyl-uronic acid-(1→4)-2-deoxy-2-sulfamido-β-D-glucopyranose disodium salt
5: 4-deoxy-α-L-threo-hex-enepyranosyluronic acid-(1→4)-2-deoxy-2-sulfamido-α-D-glucopyranosyl sodium salt
6: 4-deoxy-α-L-threo-hex-enepyranosyluronic acid-(1→4)-2-deoxy-2-acetamido-6-O-sulfo-α-D-gluco-pyranosyl disodium salt
7: 4-deoxy-α-L-threo-hex-4-enepyranosyluronic acid-(1→4)-1,6-anhydro-2-deoxy-2-sulfamido-β-D-glucopyranose disodium salt (disaccharide 1)
β: 4-deoxy-α-L-threo-hex-4-enepyranosyluronic acid-(1→4)-1,6-anhydro-2-deoxy-2-sulfamido-β-D-mannopyranose disodium salt (disaccharide 2)
9: 4-deoxy-2-O-sulfo-α-L-threo-hex-enepyranosyl-uronic acid-(1→4)-2-deoxy-2-acetamido-α-D-glucopyranosyl disodium salt
10: 4-deoxy-α-L-threo-hex-4-enegalactopyranosyl-uronic acid-(1-44)-2-deoxy-2-sulfamido-6-O-sulfo-β-D-glucopyranose trisodium salt
11: 4-deoxy-α-L-threo-hex-enepyranosyluronic acid-(1→4)-2-deoxy-2-sulfamido-6-O-sulfo-β-D-glucopyranosyl trisodium salt
12: 4-deoxy-2-O-sulfo-α-L-threo-hex-enepyranosyl-uronic acid-(1→4)-2-deoxy-2-sulfamido-α-D-glucopyranosyl trisodium salt
13: 4-deoxy-2-O-sulfo-α-L-threo-hex-4-enepyranosyl-uronic acid-(1→4)-1,6-anhydro-2-deoxy-2-sulfamido-β-D-glucopyranose trisodium salt (Disaccharide 3)
14: 4-deoxy-2-O-sulfo-α-L-threo-hex-enepyranosyl-uronic acid-(1→4)-2-deoxy-2-acetamido-6-O-sulfo-β-D-glucopyranosyl trisodium salt
15: 4-deoxy-α-L-threo-hex-enepyranosyluronic acid-(1→4)-2-deoxy-2-acetamido-6-O-sulfo-α-D-gluco-pyranosyl-(1→4)-β-D-glucopyranosyluronic acid-(1→4)-2-deoxy-2-sulfamido-3-O-sulfo-α-D-gluco-pyranosyl)pentasodium salt
16: 4-deoxy-2-O-sulfo-α-L-threo-hex-enepyranosyl-uronic acid-(1→4)-2-deoxy-2-sulfamido-6-O-sulfo-α-D-glucopyranosyl tetrasodium salt
17: 4-deoxy-α-L-threo-hex-enepyranosyluronic acid-(1→4)-2-deoxy-2-acetamido-6-O-sulfo-α-D-gluco-pyranosyl-(1→4)-β-D-glucopyranosyluronic acid-(1→4)-2-deoxy-2-sulfamido-3,6-di-O-sulfo-α-D-glucopyranosyl)hexasodium salt
18: 4-deoxy-2-O-sulfo-α-L-threo-hex-enepyranosyl-uronic acid-(14)-2-deoxy-2-sulfamido-6-O-sulfo-D-glucopyranosyl-(1→4)-2-O-sulfo-α-L-idopyranosyluronic acid hexasodium salt
19: 4-deoxy-2-O-sulfo-α-L-threo-hex-enepyranosyl-uronic acid-(1→4)-2-deoxy-2-sulfamido-6-O-sulfo-α-D-glucopyranosyl-(1→4)-2-O-sulfo-α-L-idopyranosyluronic acid-(1→4)-1,6-anhydro-2-deoxy-sulfamido-β-D-mannopyranose, hexasodium salt (tetrasaccharide 1)
20: 4-deoxy-α-L-threo-hex-enepyranosyluronic acid-(1→4)-2-deoxy-2-acetamido-α-D-glucitol sodium salt
21: 4-deoxy-α-L-threo-hex-enepyranosyluronic acid-(1→4)-2-deoxy-2-sulfamido-β-D-glucitol disodium salt
22: 4-deoxy-α-L-threo-hex-enepyranosyluronic acid-(1→4)-2-deoxy-2-sulfamido-α-D-glucitol disodium salt
23: 4-deoxy-α-L-threo-hex-enepyranosyluronic acid-(1→4)-2-deoxy-2-acetamido-6-O-sulfo-α-D-glucitol disodium salt
24: 4-deoxy-2-O-sulfo-α-L-threo-hex-enepyranosyl-uronic acid-(1→4)-2-deoxy-2-acetamido-α-D-glucitol disodium salt
25: 4-deoxy-α-L-threo-hex-enegalactopyranosyluronic acid-(1→4)-2-deoxy-2-sulfamido-6-O-sulfo-β-D-glucitol trisodium salt
26: 4-deoxy-α-L-threo-hex-enepyranosyluronic acid-(1→4)-2-deoxy-2-sulfamido-6-O-sulfo-α-D-glucitol trisodium salt
27: 4-deoxy-2-O-sulfo-α-L-threo-hex-enepyranosyl-uronic acid-(1→4)-2-deoxy-2-sulfamido-α-D-glucitol trisodium salt
28: 4-deoxy-2-O-sulfo-α-L-threo-hex-enepyranosyl-uronic acid-(1→4)-2-deoxy-2-acetamido-6-O-sulfo-α-D-glucitol trisodium salt
29: 4-deoxy-α-L-threo-hex-enepyranosyluronic acid-(1→4)-2-deoxy-2-acetamido-6-O-sulfo-α-D-gluco-pyranosyl-(1→4)-β-D-glucopyranosyluronic acid-(1→4)-2-deoxy-2-sulfamido-3-O-sulfo-α-D-glucitol) pentasodium salt
30: 4-deoxy-2-O-sulfo-α-L-threo-hex-enepyranosyl-uronic acid-(1→4)-2-deoxy-2-sulfamido-6-O-sulfo-α-D-glucitol trisodium salt
31: 4-deoxy-α-L-threo-hex-enepyranosyluronic acid-(1→4)-2-deoxy-2-acetamido-6-O-sulfo-α-D-glucopyranosyl-(14)-β-D-glucopyranosyl-uronic acid-(1→4)-2-deoxy-2-sulfamido-3,6-di-O-sulfo-α-D-glucitol) hexasodium salt
32: 4-deoxy-2-O-sulfo-α-L-threo-hex-enepyranosyl-uronic acid-(1→4)-2-deoxy-2-sulfamido-6-O-sulfo-α-D-glucopyranosyl-(14)-2-O-sulfo-α-L-idopyranosyluronic acid hexasodium salt (form reduced with NaBH₄).

1. A method for analysing heparins or low-molecular-weight heparins, comprising:
1—depolymerizing the sample by the action of heparinases;
2—optionally, reducing the depolymerized sample; and
3—assaying by high-performance liquid chromatography.

2. The method as claimed in claim 1, further comprising carrying out a search for the presence of oligosaccharide chains whose end is modified with a 1,6-anhydro bond.

3. The method as defined in claim 1, wherein the heparinases are in the form of a mixture of heparinase 1 (EC 4.2.2.7.), heparinase 2 (heparin lyase II) and heparinase 3 (EC s4.2.2.8.).

4. The method as defined in claim 1, wherein the heparin depolymerized by the action of heparinase (depolymerizate) is then subjected to a reducing agent.

5. The method as defined in claim 4, wherein the reducing agent is NaBH₄ or an alkali metal salt of the borohydride anion.

6. The method as defined in claim 1 wherein the low-molecular weight heparin is enoxaparin.

7. The method as defined in claim 1, in which the chromatographic method used is an anion-exchange chromatography.

8. The method as defined in claim 7, further comprising a mobile phase which is transparent in the UV region up to 200 nm.

9. The method as defined in claim 8, wherein the mobile phase used is sodium perchlorate, methanesulfonate salts or phosphate salts.

10. The method as defined in claim 7, wherein said method can selectively detect acetylated sugars.

11. The method as defined in claim 10, wherein the selective detection of the acetylated sugars is carried out taking as signal the difference between the absorbance at two wavelengths chosen such that the absorptivity of the nonacetylated saccharides cancels out.

12. The method as claimed in claim 1, wherein the 1,6-anhydro residues obtained during the depolymerization reaction are the following:

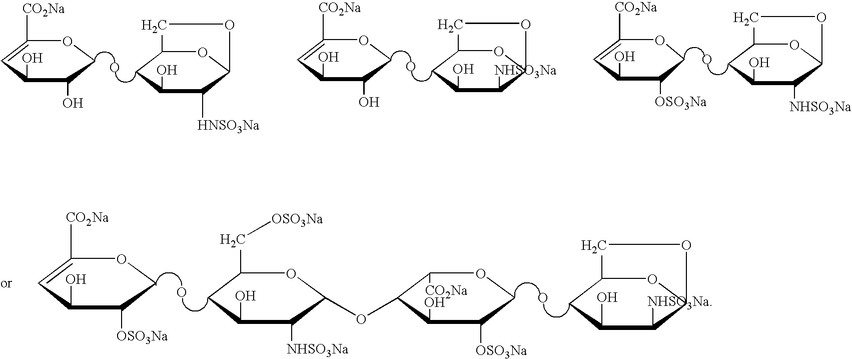

13. A 1,6-anhydro derivative of formula (disaccharide 1)

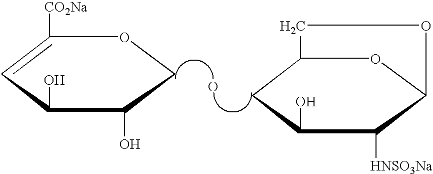

14. A 1,6-anhydro derivative of formula (disaccharide 2)

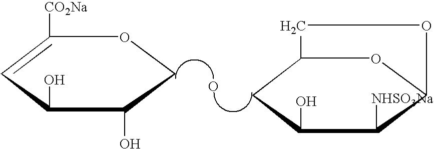

15. A 1,6-anhydro derivative of formula (disaccharide 3)

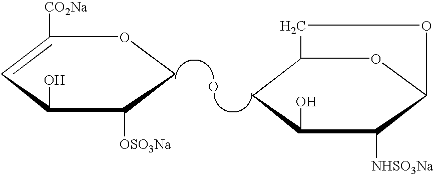

16. A trisaccharide derivative of formula: (trisaccharide 1) [chemical structure]
